(19) United States
(12) Patent Application Publication
(10) Pub. No.: US 2025/0186838 A1
(43) Pub. Date: Jun. 12, 2025

(54) CONFIGURATION OF HARDWARE TO ENABLE MULTI-MODAL FUNCTIONAL EXERCISE PROGRAMS AT DISTRIBUTED LOCATIONS

(71) Applicant: Hit Tekk Pty Ltd, Queenscliff, NSW (AU)

(72) Inventors: Mat Graham, Queenscliff, NSW (AU); Matthew Ward, Queenscliff, NSW (AU)

(21) Appl. No.: 18/845,800

(22) PCT Filed: Mar. 10, 2023
(86) PCT No.: PCT/AU2023/050172

§ 371 Date: Sep. 10, 2024

(30) Foreign Application Priority Data
Mar. 10, 2022 (AU) 2022900585
May 18, 2022 (WO) PCT/AU2022/050480

(51) Int. Cl. A63B 24/00

(52) U.S. Cl. CPC A63B 24/0075; A63B 24/0006; A63B 24/0062; A63B 2024/0071; A63B 2024/0078

(57) ABSTRACT

Configuration of hardware enables multi-modal functional exercise programs at distributed locations. For example, systems, software, methods and hardware are associated with such configuration. Some embodiments relate to a use-case whereby a plurality of exercise platform devices are each connected to a network, each machine being configured to enable multi-modal functional workouts (for example, including medicine ball workouts, cardiovascular workouts, and/or resistance training).

### CROSS-REFERENCE TO RELATED APPLICATIONS

This application is a national phase entry under 35 U.S.C. § 371 of International Patent Application PCT/AU2023/050172, filed Mar. 10, 2023, designating the United States of America and published as International Patent Publication WO 2023/168498 A1 on Sep. 14, 2023, which claims the benefit under Article 8 of the Patent Cooperation Treaty of Australian Patent Application Serial No. 2022900585, filed Mar. 10, 2022, and International Patent Application No. PCT/AU2022/050480, filed May 18, 2022.

### TECHNICAL FIELD

The present disclosure relates, in various embodiments, to configuration of hardware to enable multi-modal functional exercise programs at distributed locations. For example, embodiments include systems, software, methods and hardware associated with such configuration. Some embodiments relate to a use-case whereby each of a plurality of exercise platform devices are connected to a network, each machine being configured to enable multi-modal functional workouts (for example, including medicine ball workouts, cardiovascular workouts, and/or resistance training). Although the present disclosure is described primarily in connection with such examples, it will be appreciated that further embodiments find wider application.

### BACKGROUND

Any discussion of the background art throughout the specification should in no way be considered as an admission that such art is widely known or forms part of common general knowledge in the field.

Functional training workouts have become increasingly popular in recent years. These commonly involve “circuit-style” workouts, whereby a participant engages in a range of different exercises in a prescribed sequence, typically involving the use of a range of different pieces of exercise equipment (including the likes of medicine balls, kettle bells, rowing machines, stationary bikes, and the like). Given the multi-modal nature of these workouts, it is challenging to monitor participant exercise metrics (beyond physiological parameters such as heart rate). These challenges are compounded where a participant wishes to participate in a workout outside a conventional gymnasium environment.

It is an object of the present disclosure to overcome or ameliorate at least one of the disadvantages of the prior art, or to provide a useful alternative.

### BRIEF SUMMARY

Example embodiments are described below in the section entitled “claims,” and in the section entitled “detailed description.”

One embodiment provides a system configured to enable multiple-activity functional workouts via distributed exercise machines, the system including:
a machine configuration database that contains a plurality of modality data sets, wherein each modality data set includes, for a specific exercise modality, data representative of:
device configuration data executable via a given one of the exercise machines thereby to configure that device to receive physical input representative of the specific exercise modality;
processing configuration data executable via a that one of the exercise machines thereby to configure the device to process physical input representative of the specific exercise modality when operated in accordance with the device configuration data;
a playlist selector module, wherein the playlist selector module is configured to enable selection of a defined playlist, wherein each playlist is representative of instructions to deliver a plurality of exercise modalities in a predefined sequence;
a playlist delivery module, wherein the playlist delivery module is responsive to a selection via the playlist selector module thereby to cause delivery of data via a network to a defined one or more of the distributed exercises devices, thereby to configure those devices to enable execution of the selected playlist.

One embodiment provides a computer implemented method configured to enable content generation for a system that enables multiple-activity functional workouts via distributed exercise machines, the method including:
maintaining data representative of customizable templates for a plurality of exercise modalities;
providing a user interface that enables a first user to generate a playlist including a plurality of different selected exercise modalities;
providing access via the user interface for the first user to set values for a plurality of variables in respect of respective customizable templates for each of the selected exercise modalities, such that these are associated with the generated playlist;
defining a unique identification code for the generated playlist; and
enabling a second user to: (i) identify the generated playlist; (ii) select the generated playlist; and (iii) provide an instruction to download data associated with the generated playlist to a specific one of the distributed exercise machines designated by the second user, thereby to configure that specific one of the distributed exercise machines to download data associated with the playlist and guide the second user through a multi-modal workout defined by the playlist.

Reference throughout this specification to “one embodiment,” “some embodiments” or “an embodiment” means that a particular feature, structure or characteristic described in connection with the embodiment is included in at least one embodiment of the present disclosure. Thus, appearances of the phrases “in one embodiment,” “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

### BRIEF DESCRIPTION OF THE DRAWINGS

Embodiments of the present disclosure will now be described, by way of example only, with reference to the accompanying drawings.

FIG. 1 illustrates a technology framework according to one embodiment.

FIG. 2 illustrates an example workout device according to one embodiment.

FIG. 3A illustrates a method according to one embodiment.

FIG. 3B illustrates a method according to one embodiment.

FIGS. 4A-4C illustrate an example multi-modal workout machine according to an embodiment.

### DETAILED DESCRIPTION

The present disclosure relates, in various embodiments, to configuration of hardware to enable multi-modal functional exercise programs at distributed locations. For example, embodiments include systems, software, methods and hardware associated with such configuration. Some embodiments relate to a use-case whereby a plurality of exercise platform devices are each connected to a network, each machine being configured to enable multi-modal functional workouts (for example, including medicine ball workouts, cardiovascular workouts, and/or resistance training). Although the present disclosure is described primarily in connection with such examples, it will be appreciated that further embodiments find wider application.

In overview, technology disclosed herein is directed to the authoring and delivery of multi-modal workouts via distributed networked exercise machines. It has been identified that, by providing a data structure that combines aspects of machine configuration and digital signal processing, there is an ability to configure end-user machines to deliver a range of multi-modal functional workouts, including monitoring of physical performance metrics.

The term “multi-modal workout,” as described herein, refers to a workout that includes multiple different forms of human body movement, which are measured (including in terms of workout intensity/effort) via sensor components. Examples include: (i) body weight exercises, such as push-ups; (ii) footwork exercises such as skipping and jumping; (iii) resistance exercises utilizing resistance bands; (iv) free weight exercise, for example, various kettle bell motions; and (v) impact exercise such as medicine ball. It will be appreciated that, depending on the nature of exercise machine present for the purposes of a given embodiment, other forms of human body movement may be supported. In some embodiments, a hardware unit is configured to monitor multiple workout modalities using common sensor hardware, for example, pressure sensors, and time series data from those sensors is processed differentially based on a form of exercise that is to be performed.

Embodiments are described by reference to a “workout platform machine” as an exemplary piece of multi-modal workout hardware. It should be appreciated that this is an example only, and that the technology herein may be applied across a wide range of exercise machines that are able to be controlled thereby to deliver multi-modal workouts.

An example of a “workout platform machine” is disclosed in PCT patent application PCT/AU2022/050480, which is incorporated herein by this reference, and embodiments described below. In brief, a “workout platform machine” is a piece of equipment that includes sensors configured to measure force applied to a platform, for example, as a time series data. This data is then processed by various means, for example, using digital signal processing techniques, thereby to quantify exercise metrics. For example, exercise metrics may include the likes of: (i) whether a particular exercise has been performed; (ii) a metric representative of effort for that exercise; (ii) a metric representative of cumulative effort for multiple exercise repetitions; (iii) metrics representative of tempo for an exercise repetition, or component of an exercise repetition; and (iv) metrics representative of a rate at which repetitions are performed. Other metrics may be used. An example multi-modal workout platform machine is configured to measure force applied by a user both impact-based exercises (e.g., slam balls, jumping, running, etc.) and resistance-based exercises (for example, where resistance bands accentuate fore between a user and the platform, as measurable by sensors).

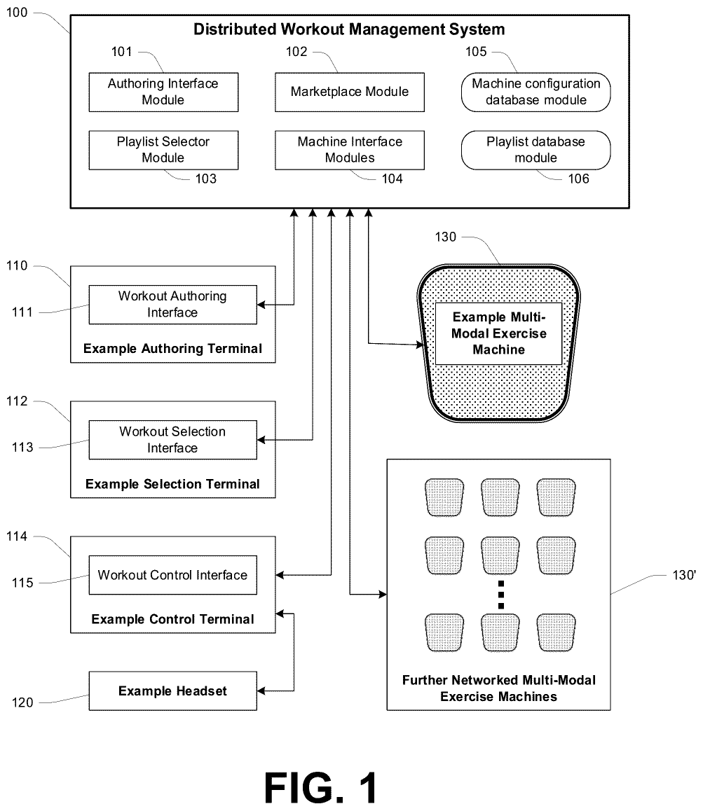

Some embodiments include a system configured to enable multiple-activity functional workouts via distributed exercise machines. An example is described below by reference to FIG. 1, which illustrates a distributed workout management system 100. System 100 may be defined by one or a plurality of server components (which may be distributed across a plurality of computing systems). For the present purposes, system 100 is illustrated and described by reference to functional components, including “modules.”

As used here, the term “module” refers to a software component that is logically/notionally separable (a computer program), or a hardware component. The module of the embodiment refers to not only a module in the computer program but also a module in a hardware configuration. The discussion of the embodiment also serves as the discussion of computer programs for causing the modules to function (including a program that causes a computer to execute each step, a program that causes the computer to function as means, and a program that causes the computer to implement each function), and as the discussion of a system and a method. For convenience of explanation, the phrases “stores information,” “causes information to be stored,” and other phrases equivalent thereto are used. If the embodiment is a computer program, these phrases are intended to express “causes a memory device to store information” or “controls a memory device to cause the memory device to store information.” The modules may correspond to the functions in a one-to-one correspondence. In a software implementation, one module may form one program or multiple modules may form one program. One module may form multiple programs. Multiple modules may be executed by a single computer. A single module may be executed by multiple computers in a distributed environment or a parallel environment. One module may include another module. In the discussion that follows, the term “connection” refers to not only a physical connection but also a logical connection (such as an exchange of data, instructions, and data reference relationship). The term “predetermined” means that something is decided in advance of a process of interest. The term “predetermined” is thus intended to refer to something that is decided in advance of a process of interest in the embodiment. Even after a process in the embodiment has started, the term “predetermined” refers to something that is decided in advance of a process of interest depending on a condition or a status of the embodiment at the present point of time or depending on a condition or status heretofore continuing down to the present point of time. If “predetermined values” are plural, the predetermined values may be different from each other, or two or more of the predetermined values (including all the values) may be equal to each other. A statement that “if A, B is to be performed” is intended to mean “that it is determined whether something is A, and that if something is determined as A, an action B is to be carried out.” The statement becomes meaningless if the determination as to whether something is A is not performed.

The term “system” refers to an arrangement where multiple computers, hardware configurations, and devices are interconnected via a communication network (including a one-to-one communication connection). The term “system,” and the term “device,” also refer to an arrangement that includes a single computer, a hardware configuration, and a device. The system does not include a social system that is a social “arrangement” formulated by humans.

At each process performed by a module, or at one of the processes performed by a module, information as a process target is read from a memory device, the information is then processed, and the process results are written onto the memory device. A description related to the reading of the information from the memory device prior to the process and the writing of the processed information onto the memory device subsequent to the process may be omitted as appropriate. The memory devices may include a hard disk, a random-access memory (RAM), an external storage medium, a memory device connected via a communication network, and a ledger within a CPU (Central Processing Unit).

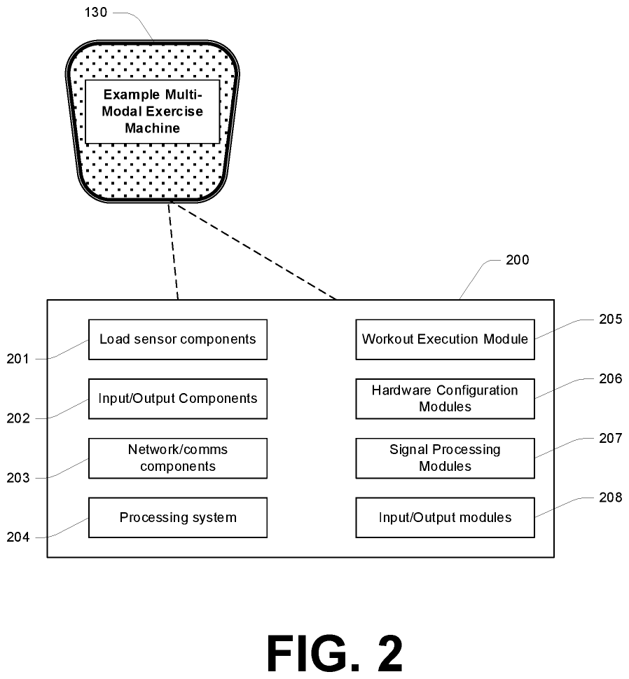

The example multi-modal exercise machine 130 of FIG. 2 includes one or more load sensor components 201. This may include multiple load sensors, which collectively provide a single signal (e.g., via an averaging algorithm), and/or load sensors having varying sensitivity ratings (e.g., one tuned for relatively lower forces, one tuned for relatedly higher forces). The multi-modal exercise machine 130 additionally includes various input/output components 202, for example, including buttons, lights, speakers, audio outputs (including Bluetooth), connection to peripherals (for example, wearable devices such as heart rate monitors) and so on. Network/communications components 203 allow multi-modal exercise machine 130 to communicate with other devices, including devices provided via system 100. A processing system 204 coordinates machine functions, including delivery of workouts and processing of metrics (e.g., processing of time-series data delivered by load sensor components 201). A workout execution module 205 is configured to execute data representative of a workout playlist, as discussed in more detail further below. Execution of the workout playlist causes operation of hardware configuration modules 206 thereby to configure load sensor components 201 and/or other hardware specific to given exercise modalities, and/or operation of signal processing modules 207 to process time-series data from load sensors components 201 and/or other hardware in a manner specific to a given exercise modality. Input/output modules 208 control processing if inputs and delivery of signals via input/output components 202.

System 100 is configured to communicate via a network with a plurality of exercise devices, which in this example are workout platform machines. FIG. 1 illustrates an example multi-modal exercise machine 130, and a plurality of further networked multi-modal exercise machines 130′. These communicate via the Internet and/or local networks.

System 100 is also configured to communicate with a plurality of remote terminals, which may include, for example, the likes of smartphones, tablets, PCs, laptops, gaming consoles and the like. Each of these terminals is configured to render a user interface that allows a user to interact with functionalities made available via system 100. For example, the rendered user interface may be provided via a locally executing software application, via web code executed within a local web browser application, or via other means. Three example categories of terminal are illustrated in FIG. 1. These are distinguished by function for the purposes of illustration. In some embodiments, a common terminal (such as a smartphone) performs the role of two or more of these terminals (optionally via a single software application). The illustrated example terminals described below, with reference to their interaction with system 100:

An example authoring terminal 110, which renders a workout authoring interface. This workout authoring interface enables a user to define a “playlist” that includes a plurality of exercises of different modalities. The authoring interface preferably allows a user to set parameters for each of the exercises, and/or a manner in which they are sequenced together. This is described in more detail further below. In overview, an output of the workout authoring interface is a workout playlist, which is able to be executed by a given one or more of machines 130/130′. Workout playlist data is stored by system 100 via a playlist database module 106.

System 100 provides an authoring interface module, which provides back-end functionality to authoring interface 111. This includes:
Maintaining data representative of customizable templates for a plurality of exercise modalities. This allows a user to select an exercise modality (for example, “bent over row”) and customize a template by inputting values for available variables. Available variables may differ between modalities. Generic examples include number of repetitions; target work rate for each repetition; intra-repetition tempo (optionally including positive motion, negative motion, and holds); inter-repetition tempo; repetition number; exercise duration; and the like. In some cases more complex variables are set, for example, variables relating to form (e.g., explosive power vs movement control).
Enabling a user to define a sequence for customized templates. This may include ordering/numbers of sets, and optionally standard workout type characteristics (e.g., ladder progressions, Tabata style training, AMRAP sets, and the like).
Enabling a user to define/customize rules for the delivery of audible/visual signals to a user (for example, thereby to provide instructions and/or feedback). This may include a rules-based approach whereby a user selects a condition (e.g., “before commencement of exercise,” “upon detection of successful repetition,” “if repetition rate falls below X RPM,” “when approximately 30 seconds before next modality”) and a signal (for example, an instruction signal with audible verbal explanation of how to perform that modality or the next modality, feedback on performance, and so on).
Processing data generated via the authoring of customized templates, sequencing, and signal delivery rules thereby to define executable data for a workout playlist that can be delivered to one or more of machines 130/130′ and executed via that/those machines.

In some embodiments, the authoring interface is supplemented by or replaced by a more complex coding/development interface, which provides additional flexibility over workout authoring, feedback, and the like. For example, this may be used for development of premium content that is sold on a marketplace (discussed further below).

An example selection terminal 112, which renders a workout selection interface 113. Workout selection interface 113 enables a user to select a workout playlist from a selection of predefined workout playlists made available via a playlist database module 106. The nature of the selection interface varies between embodiments. Optional functionalities include: (i) user-based filtering, whereby a user is able to view workout playlists created by specified users; (ii) workout type filtering, whereby a user is able to search/browse workout paylists based on attributes; (iii) subscriptions, whereby a user is presented with workouts based on a subscription, for example, allowing for display of “workout of the day”; and (iv) a marketplace whereby a user is enabled to browse and purchase workout playlist content, including individual playlists and/or subscriptions to playlists from a given creator. The workout selection interface is configured to enable selection of a workout, and the delivery of that workout for execution at a specified one or more of machines 130/130′. For example, this may include either or both of: (i) a user selecting a workout playlist for delivery to a specified machine, so that they can use that machine; and (ii) a user selecting a workout playlist for deliver to a plurality of specified machines, so that a plurality of users can use that workout playlist. The latter may be synchronous (e.g., a trainer configures multiple machines in a facility such that users can participate in a common workout playlist as a group) or asynchronous (e.g., a trainer sends a daily playlist to a plurality of machines associated with his/her clients).

System 100 provides a playlist selector module 103 that facilitates searching/browsing of available workout playlists by reference to a playlist database module 106. Optionally, a marketplace module 102 allows a user to purchase premium content for a price (optionally including individual workout playlists, collections of workout playlists, and/or a subscription to workout playlists from a specific source, for example, a particular persona; trainer or the like). The playlist selector module 103 is configured to enable selection of a defined playlist. Upon selection, a machine interface module 104 provides a playlist delivery module that is responsive to a selection via the playlist selector module 103 thereby to cause delivery of data via a network to a defined one or more of the distributed exercises devices, thereby to configure those devices to enable execution of the selected playlist.

In terms of creating a nexus between a given workout selection interface and one or more specific workout machines 130/130′, various approaches may be used. For example, each machine may have a unique code (which may, for example, be read optically, via NFC/BLE, via alphanumeric input, and the like), thereby facilitating identification of a machine via a selection terminal, and addressing of data to that machine via system 100. In some embodiments, workout selection interface 113 allows a user to create a set of “favorite” machines (which are optionally able to be formed into groups), thereby to streamline a process by which a desired one or more machines are selected for the purposes of machine interface module 104.

An example control terminal 114, which renders a workout control interface 115. This facilitates the engagement between a user and a platform machine during the execution of a workout playlist. For example, in some embodiments, workout control interface 115 provides instructions, for example, audible instructions via a headset 120, to a user of a machine thereby to guide the user through physical activities associated with an executing workout playlist. Preferably workout control interface 115 additionally communicates metrics representative of a user's performance in undertaking a workout. This may be intra-workout (for example, a rules engine associates observed metrics with audible output messages, such that a user receives real-time personal trainer style input on their performance) and/or retrospective (for example, a user receives input as to their performance following completion of an exercise/set/workout). In alternate embodiments, terminal 115 and/or headset 120 communicates directly with example multi-modal exercise machine 130 (e.g., via Bluetooth), rather than communication routed via machine interface modules 104 of system 100.

In combination, interfaces 111, 113 and 115 enable a process of workout playlist authoring, workout playlist selection, and workout playlist execution (including delivery of performance metrics).

A key function of system 100 is the configuration of each of machines 130/130′ to execute selected workout playlists. Unlike conventional media distribution arrangements (e.g., streaming of audio/video and/or distribution of media files), system 100 requires functionality to provide instructions to the individual machines such that they are configured to perform any one or more of the following:
(i) Configure internal hardware thereby to collect sensor data relevant to a specific exercise modality. For example, this may include setting sensitivity thresholds for sensor components, sampling rates, hardware component activation/deactivation, and the like.
(ii) Configure processing components to process sensor data for each specific exercise modality. For example, this includes executable instructions by which an input signal from a sensor (for example, a time-series signal representative of force) is processed thereby to identify data artefacts specific to a given exercise. In practical terms, this may include differentiating a push-up motion from a resistance band rowing motion, identifying components of a medicine ball lift/hod/slam motion, and so on. This is preferably used both for the purposes of identifying that an exercise has been performed (e.g., does the time-series data match a predefined exercise “fingerprint,” based on DSP method and/or operation of an AI classifier) and determining exercise metrics (which will be different for each exercise/modality, and optionally include quantification of components of the time-series data based on varying processing algorithms).
iii) Configure user interface components to deliver instructions (e.g., visual and/or audible) to a user thereby to provide instructions and/or feedback in relation to exercises defined by a workout playlist.

In this regard, system 100 includes a machine configuration database module 105. This provides access to a database that contains a plurality of modality data sets. Each modality data set includes, for a specific exercise modality, data representative of:
(i) Device configuration data executable via a given one of the exercise machines thereby to configure that device to receive physical input representative of the specific exercise modality. For example, by referencing a particular modality set, system 100 is able to instruct a given machine with respect to how it should configure its hardware thereby to monitor a particular modality within a given workout playlist. The device configuration data is preferably stored at a cloud location, and downloaded to individual machines as needed.
(ii) Processing configuration data executable via a that one of the exercise machines thereby to configure the device to process physical input representative of the specific exercise modality when operated in accordance with the device configuration data. This allows for the machine to determine whether a given exercise repetition has been physically performed (which may include “not performed” or “incorrectly performed”), and determine metrics associated with performance (which may include non-performance and/or incorrect performance).
(iii) UI configuration data, thereby to enable a device to deliver via available user interface hardware (e.g., a screen, lights, etc.) instructions and/or feedback in relation to exercises defined by a workout playlist.

The processing configuration data may include (or be associated with) a machine output protocol. A “machine output protocol” is a set of logic/rules that governs the manner in which output is delivered to a user during the performance of a workout playlist. For example, rules may be defined during authoring including the likes of:
A rule to provide a signal (visual and/or audible) when a repetition is correctly performed. For example, this can be used as a counter.
A rule to provide a signal (visual and/or audible) when a repetition is incorrectly performed. For example, this can be used to inform a user that they are performing an exercise incorrectly.
A rule to provide a signal (visual and/or audible) when a repetition is correctly performed with metrics within a predefined range. For example, this may be used to inform a user if they are performing an exercise at a prescribed intra-repetition prescribed tempo, and/or inform a user in relation to the power/effort being exerted during a repetition.
A rule to provide a signal (visual and/or audible) when cumulative metrics move into (or out of) a predefined range. For example, this may be used to provide feedback regarding repetition rate, inter-repetition tempo, power/effort decrease over time, and the like.
A rule to provide a signal where metrics for a given exercise includes defined data attributes. For example, this may be used to identify incorrect form during a free-weight motion (e.g., jerky motions).
A rule to provide a signal where a certain proportion of repetitions (or other completion metric) is achieved, thereby to trigger delivery of information/instructions for a next exercise modality in an executing workout playlist.

Via such an approach, a workout playlist is able to be authored and executed such that visual and/or audible signals are provided to a user based on monitoring of data collected via machine sensors, enabling automated instructions, adaptive feedback, and the like.

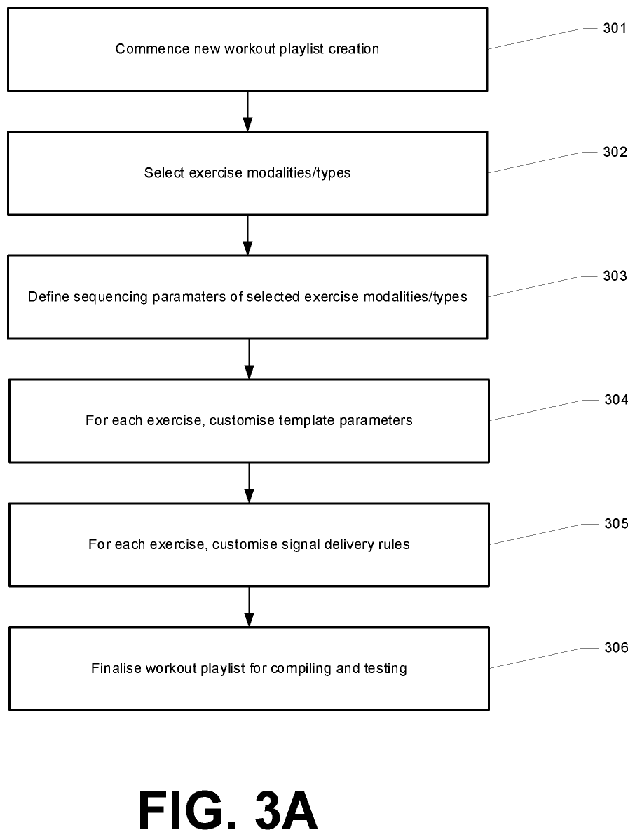

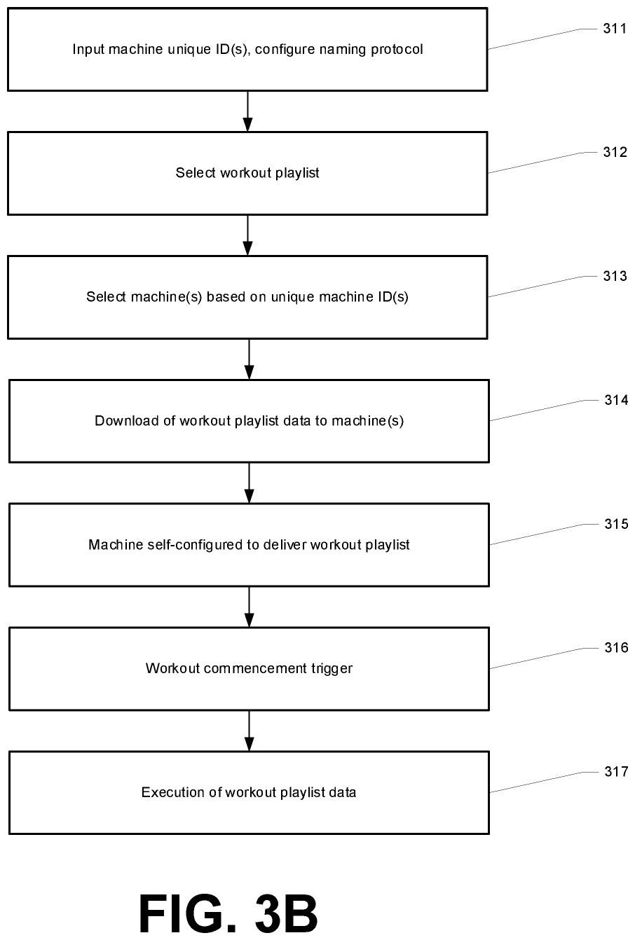

FIG. 3A illustrates an example workout playlist authoring method, for example, performed based on interaction between interface 111 and module 101. It will be appreciated that this is an example only, and provided for the sake of context. Various modifications to this method are made for the purposes of further embodiments.

Block 301 represents a process including commencing generation of a new workout playlist. This may include defining a workout name/ID via the authoring interface.

Block 302 represents a process whereby the authoring interface displays available exercise modalities/types, which are preferably navigable via a search/browse/filter arrangement. A user selects desired modalities/types, and orders them into a sequence (for example, by graphical manipulation of icons), as represented by block 303. The same modality/type may be selected on multiple occasions, such that it can be customized with different metrics (e.g., repetition numbers, set time, etc.).

The level of granularity by which modalities/types are defined varies between embodiments, and hierarchical structures may be used. In that regard, each exercise modality/type is associated with a data set in the machine configuration database, which provides a customizable template by which that modality/type is customized by setting of parameters. So, for instance, there may be a modality type of “resistance band” with first level subtype of “bent over row” and second level sub types of “strength,” “power,” “cardio” and “control.” Each of those is associated with a template having customizable variables. For example, “strength” may have key variables associated with requirements for objective peak load exerted via an exercise, “power” may have key variables associated with peak load and tempo, “cardio” may have key variables associated with repetition rate, and “control” may have key variables associated with intra-exercise tempo and motion smoothness.

Block 304 represents a process by which template parameters are customized for each modality/type in the defined sequence. This may include accessing default parameters. Otherwise, a user is presented with each of the available customizable parameters, and sets a variable value for each.

Block 305 represents a process including customization of signal delivery rules. Preferably, each template has default rules, and a user modifies those (for example, with custom audio recordings) and/or adds additional rules (for example, custom audio when a particular data artefact is observed, for example, RPM dropping below a defined threshold). In some embodiments, a user is able to search for non-standard metrics that can be measured during the exercise modality/type, and define signals based on those. Signals may be both real-time instructive, and/or representative of retrospective performance (for example, to allow a user to assess their performance, optionally relative to other persons who have completed the same workout).

Block 306 represents a process whereby the paylist is compiled, which includes system 100 defining device configuration data, processing configuration data, and other executable instructions that are provided to a given one or more of machines 130/130′ thereby to enable those machines to deliver the workout playlist. In some embodiments, this includes a function to allow for a virtual simulation of the playlist for the purposes of testing.

FIG. 3B shows an example method for delivering a workout playlist via one or more machines. Block 311 represents a process inputting of unique machine IDs. This may include any one or more of: manual input of an alphanumeric code; scanning of an optically readable code (such as a QR code); scanning a radio-frequency code (for example, RFID or NFC); discovery via Bluetooth/BLE technologies; and other such approaches. Optionally a machine name is displayed/defined (for example, based on the location of the machine, such as “GYM FLOOR #3,” “GENERIC HOTEL SYDNEY, ROOM 301,” or based on an owner's name). Optionally a user is able to create machine groups, such as “ALL GYM FLOOR MACHINES,” or “MY REMOTE PERSONAL TRAINING CLIENTS.” Block 312 represents a process of selecting a playlist, for example, based on a search/browse functionality. Block 313 represents selection of a machine/machines. For example:
In one example a user scans a machine prior to selection, and that machine is selected by default.
In another example a user is prompted to scan a machine after selection, and that machine is selected.
In another example a trainer user selects a group of machines, an sends the selected workout playlist to all of those machines (for example, to enable communal synchronous workout in a common exercise space, or to configure multiple remote machines with a “workout of the day”).

Block 314 represents downloading of data representative of the selected workout playlist to the selected machine/machines. In some cases a machine is able to locally store multiple playlists, and a user is able to select between those. As represented by block 315, a machine self-configures to deliver a playlist (e.g., by configuring sensor hardware for a first exercise, and queuing initial workout instruction signals). Block 316 represents a commencement trigger, which may be an individual machine trigger (for example, pressing a “start workout” button), or a remote trigger (thereby to trigger synchronous workouts across multiple machines, optionally with competitive metrics being displayed via a common screen). This commences playlist execution (block 317), including configuration/reconfiguration of hardware to monitor the various exercise modalities/types, and signal processing thereby to define workout metrics and deliver feedback/instructions.

It should be appreciated that the above disclosure provides advances in the field of configuration of distributed workout equipment. For example, it allows for functional multi-modal workouts to be authored and distributed across multiple locations, allowing common hardware to instruct, monitor and report on multiple different exercise modalities across a functional workout.

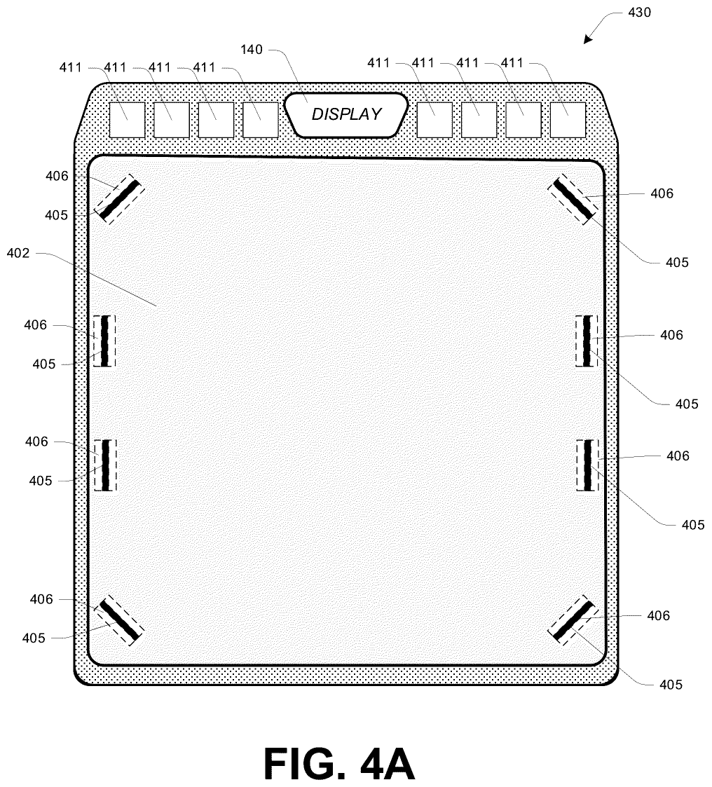

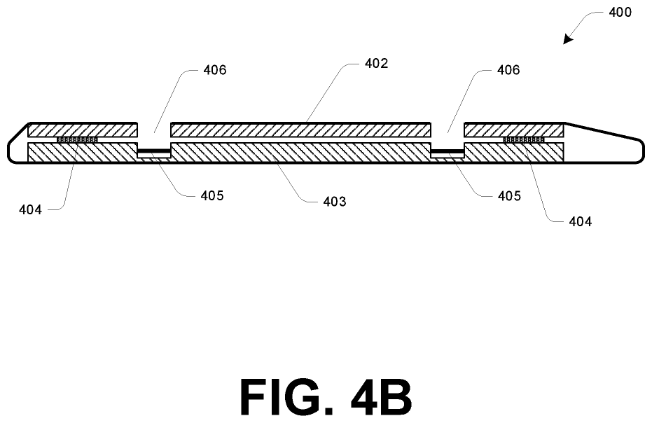

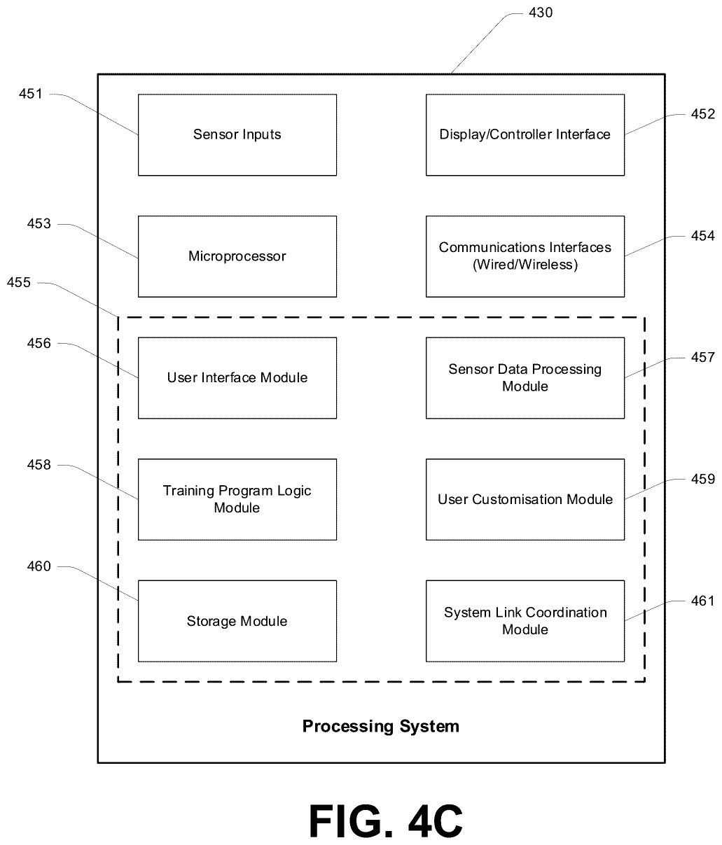

Example hardware that is optionally used in the context of embodiments described above will now be described by reference to FIGS. 4A-4C. In overview, various embodiments include a device including a platform having an upper surface that is configured to enable measurement of an applied force. For example, in some embodiments, this upper surface is configured such that a user is enabled to repeatedly impact a portable object (such as a medicine ball) onto that upper surface. The surface is preferably large enough to allow for a user to stand on the surface and perform exercises such as stationary running and/or skipping. In this regard, the upper surface is preferably substantially planar, and orientated in a substantially horizontal plane. One or more sensors configured to monitor force applied to the upper surface of the platform. These are arranged such that the sensors are configured to monitor metrics associated with one or more varieties of physical exercise. For example, in some embodiments, the one or more varieties of physical exercise include one or more of the following:
The impacting of the portable object onto the surface, for example, an impact force associated with the impacting of the object onto the surface. This may include a portable object such as a medicine ball.
The impacting of body weight onto the surface, for example, an impact force associated with body weight being applied during an exercise such as skipping, stationary running, jumping, burpees, and the like.
A combination of body weight and resistance being applied through the surface. For example, this may include an implementation where a user stands on the surface, and interacts with resistance bands (i.e., a tether configured to provide resistive force) attached the platform at a location de-coupled from the surface such that, when stretched, additional resistance force associated with the stretching is applied to the surface. In some embodiments, the platform is configured to measure time series data representative of resistance force, based on an excess force above body weight applied through the use of a resistance tether (the resistive strength of which may be known by the platform software, or unknown)/.
Combinations of the above.

It will be appreciated that these are examples only, and the hardware described herein is optionally used in a variety of other ways.

The sensors are coupled to a processing unit, which is configured to process data received from the one or more sensors, thereby to derive metrics representative of athletic performance associated with impacting a portable object onto the upper surface. These metrics optionally include:
A metric representative of force associated with a given event. The term “event” describes a set of data, for example, time series force data, which includes a peak surrounded by two troughs. Such an event may be representative of an impact event (e.g., a medicine ball impact), a human movement event (for example, a jump in the context of skipping, or a step in the context of stationary running), a resistance training event (for example, a resistance training repetition defined by a start point, resistance movement, and return to start point).
Metrics derived from time series force data during an event, for example, first and/or second order derivative data. This may be used to assess the explosiveness of movements, control during resistance training movements, repetition rates, and the like.
A metric representative of cumulative force during a workout period. For example, in the context of impact workouts, such as medicine ball workouts, this may measure a total amount of impact force generated. First and/or second order derivatives may also be used, for example, to allow calculation of work as a function of time during a workout.
A metric representative of average force during a workout period. For example, this average is calculated across a plurality of events.

A range of other metrics may also be calculated.

In this manner, the device is enabled to allow for measurement and monitoring of athletic performance during a range of workouts, including any one or more of: (i) object-impacting workouts, for example, a medicine ball workout (also known as a “slam ball” workout), a hammer slamming workout, and the like; (ii) body weight workout such as skipping, stationary running, and/or jumping; and (iii) resistance workouts, for example, resistance exercises using resistance bands.

For the sake of embodiments described below, reference is made to a particular example is an impact workout using a portable object in the form a medicine ball (with a “medicine ball” being in essence an object that is configured to be forcibly dropped with little or no rebound, for example, a sand-filled bladder). This is an example only. In some embodiments, the platform is configured to provide only for impact workouts (for instance, only medicine ball workouts); in other embodiments, the platform additionally/alternately provides for body weight and/or resistance workouts.

Three main categories of embodiment are considered herein.

The first category of embodiments provide a split platform, which includes: (i) a first set of one or more sensors configured to monitor force applied to a first region of the platform; and (ii) a second set of one or more sensors configured to monitor force applied to a second region of the platform. Typically, the first region of the platform represents an impact target region onto which the object is impacted during normal use, and the second region of the platform represents a standing region on which a user stands during normal use. This allows for segregation of force measurements that transfer through the user's feet, and force measurements that are specifically associated with the impact of the ball.

The second category of embodiments include a split platform, which includes one or more sensors configured to monitor force applied to a first region of the platform, represents an impact target region onto which the object is impacted during normal use, and a second region of the platform represents a standing region on which a user stands during normal use (which is not monitored by force sensors).

The third category of embodiments include a one-piece platform, which provides both an impact target region of the platform onto which the object is impacted during normal use, and a standing region of the platform on which a user stands during normal use (these may be only notionally defined, and not distinguishable by any stricture or feature, only by function; in some cases markings may be applied to the surface to identify a target region to assist with exercises). The one or more sensors are configured to monitor force applied to the upper surface of the one-piece platform, such that they are configured to measure force applied both an impact target region of the platform onto which the object is impacted during normal use, and a standing region of the platform on which a user stands during normal use. It will be appreciated that digital signal processing techniques may be applied to determine metrics of an impact, and optionally metrics associated with the weight of the user, and the weight of the object (noting that, during a medicine ball slamming movement, there is a period of time during which the mass of the medicine ball is separated from the platform). For example, the processing unit is configured to process a time domain signal thereby to determine a weight of the object. This time domain signal may also be processed to determine additional metrics associated with performance, for example, including a metric representative of the “explosiveness” of ball lifting. However, it will be appreciated that this category of embodiment is best suited for a range of exercise types, including body weight exercises and resistance exercises. Specifically, digital signal processing of time series force data applied to the surface is able to be processed thereby to extract metrics relevant to a wide range of exercise types.

The metrics representative of athletic performance include metrics derived from sensing of data representative of force with which the portable object is impacted on the upper surface. In the context of a medicine ball workout, these metrics may include any one or more of the following:
Workout average impact force.
Workout cumulative impact force.
Workout average repetitions-per-minute.

In some cases, one or more metrics are normalized based on the weight of a medicine ball being used, and/or the weight of the user. This is in some cases inferred from sensor data (e.g., via processing of a time-domain signal), and in other cases determined via specific input.

The upper surface may be textured or the like; the term “substantially planar” is used to indicate a generally flat region onto which an object such as a medicine ball is able to be forcibly dropped. In some embodiments, the upper surface is configured substantially in a horizontal plane. However, a tilted surface may be used (for example, to assist in return on the medicine ball to a user).

The one or more sensors configured to monitor force applied to the upper surface of the platform include at least one sensor configured to measure a metric associated with a force of the object impacting the upper surface. For example, these may include various forms of compression force transducers, load cell sensors, strain gauges, and the like. It will be appreciated that a selection of sensor will be dependent on specific design aspects, for example, location of sensors and/or configuration of a platform support. Multiple sensors may be positioned at distinct locations.

Some embodiments make use of compression force sensor, for example, sensors configured for compression force measurement with full-bridge, bonded foil strain gauge technology. These provide excellent long-term stability and ensure high performance even in applications requiring in excess of 1 million load cycles.

Some embodiments make use of strain gauges, for example, using MEMS sensor technology. In such technology, the pressure of media is measured by a silicon or silicon-on-insulator sensing element that has the piezoresistive strain gage bridge on it, with a transfer medium, typically silicon oil, present between the MEMS element and a stainless-steel diaphragm. The pressure exerted on the diaphragm is transferred to the MEMS element through the oil. The change in pressure results in a change in electrical output from MEMS elements piezoresistive strain gage. This electrical output gives us an indication of the pressure changes within the system.

Some embodiments make use of thin film sensors. In thin film sensor technology, a piezoresistive strain gauge bridge is deposited directly on the cell's stainless-steel diaphragm, which is in direct contact with the media being measured. With this technology, there is no transfer medium in between the media and sensing bridge measuring the pressure. This eliminates the damaging expansion or shrinkage that can occur in a MEMS device.

Regardless of the sensor used, the processing unit is configured to process a sensor signal, which is representative of an impact force applied to the platform, for example, being an impact force created by a user forcibly dropping/throwing a medicine ball onto the platform. Preferably, the processing unit is configured to perform a normalization process thereby to account for the weight of the medicine ball. For example, this may include subtracting ball weight from the impact force. Normalization based on ball weight may be achieved via any of the following approaches:

In some embodiments, a sensor arrangement is configured to measure force applied to a platform that is used for both standing (or assuming other poses that place all of a portion of body weight force into the platform) and object impacting. As such, this sensor arrangement provides time domain data, which varies through an athletic movement (event). For example, in the case of a medicine ball workout, this includes: (i) a period of time when the mass of the medicine ball is detectable by the sensor arrangement, for example, as it rests on the platform or is held by the user; and (ii) a period of time when the medicine ball is airborne, and hence its mass is not detectable by the sensor arrangement. Digital signal processing techniques are used to determine combined user/ball weight, user-only weight, and ball-only weight. In the case of a body weight exercise, time domain data regarding an event may include data representative a period of time during which a magnitude of force detected by the sensor progresses from a minimum, to a maximum, and back to a minimum. Rates of movement, rates of acceleration of movement, and time held at peak may all be relevant metrics. In the case of a body weight exercise, the time domain data may be processed to extract force and time metrics associated with the user pushing away from the platform (e.g., initiating a jump) and landing back on the platform. This may be processed to predict jump height, explosiveness, and the like.

In some embodiments, the device includes a weight sensor, configured to measure weight of a medicine ball in isolation (that may be the same sensor used for measuring athletic performance). This may be integrated into the platform, so that a user rests the ball on the platform initially.
In some embodiments, a user stands on a secondary platform, which contains a separate force sensor. This allows the processing unit to determine weight of a user, weight of a user with the ball, and hence weight of the ball. It will be appreciated that the measured weight varies throughout usage as the user lifts and drops the ball.
In some embodiments, a user manually enters ball weight via a user interface.
In some embodiments, a ball includes a machine-readable token (e.g., a barcode or RFID tag) which is representative of weight,

The processing unit is preferably coupled to a display screen. This display screen may be part of the device (for example, a screen provided on a vertically extending member, which is mounted to a base member of the device), or a remote display screen (for example, connected via a wired or wired configuration). The processing unit is also coupled to a user interface device, which may include a series of buttons, and/or a touch screen element provided in conjunction with the display screen.

The vertically extending member illustrated in the diagrams is exemplary only. Preferably, such a member is configured thereby to minimize risks of a user inadvertently slamming the medicine ball into a display screen and/or display screen support. In some embodiments, a display is provided on/adjacent/below platform 402, and/or projected onto such a location.

The processing unit is configured to cause the display screen to display metrics derived from the motion sensors. This may include any one or more of the following, and/or metrics derived from any one or more of the following:
Impact force (for impact exercises);
Lifting force (for resistance exercises);
Time under Force/Tension (for resistance exercises);
Cumulative impact force;
Effort, derived from impact force normalized for object mass;
Repetition rate;
Average impact force;
Average effort;
Object acceleration;
Lift force metrics;
Object acceleration;
Total mass lifted;
Calories burned; and
A performance metric based on object mass and user mass.

In some embodiments, the processing unit is configured to measure and cause displaying of a metric based on a process including: (i) determining a mass of the portable object; (ii) determining an impact force at which the portable object strikes the platform; and (iii) based on (i) and (ii), deriving a metric for effort associated with striking the object on the platform.

The processing unit, in response to commands inputted via the user interface device, is configured to execute software instructions representative of one or more predefined workout programs, thereby to, via a display screen: (i) instruct a user to perform athletic activities; and (ii) display data derived from the one or more sensors thereby to provide feedback data regarding athletic performance in a current workout program.

In some embodiments, the device includes a multi-user interface configured to enable display of relative operation of two or more of the platforms having a substantially planar upper surface and a respective one or more sensors configured to monitor force applied to the upper surface of the platform. For example, multiple devices can be linked together, with a single display screen presenting metrics. This optionally display of competitive metrics for a plurality of simultaneous users.

FIG. 4A, FIG. 4B, and FIG. 4C provide illustrations of an example device 400 according to one embodiment (a top view, side partial cutaway view, and functional diagram of processing components).

Device 400 is a standalone piece of electronic athletic training equipment, configured to enable a wide range of functional workouts. Device 400 includes a platform 402 with which a user interacts for the purpose of such workouts. Platform 402 includes an upper surface that is substantially planar, optionally including texturing via a non-slip rubber covering and/or a resilient rubber matting. The platform is preferably about 1 m×1 m, allowing ample space for a user to stand and perform various forms of functional training exercises (for example, squats, medicine ball workouts, resistance band movements, and the like).

Platform 402 is mounted to a base member 403, which are in the present embodiment connected by way of a plurality of load cells 404. These load cells are configured to measure a force applied between platform 402 and base member 403. The location and number of load calls varies between embodiments. For example, in some embodiments, between three and ten load cells are used, located a short distance inward of a periphery of platform 402, spaced around the periphery, and/or at more central locations. Force measurements at the various load cells are preferably combined, for example, via an averaging algorithm, thereby to derive data representative of a force applied downwardly on platform 402. This is preferably recorded as time-series data (i.e., force as a function of time) in a processing system 430, for example, as shown in FIG. 4C. In this regard, motion (or compressive force) of platform 402 relative to base member 403 may be used to measure force exerted into platform 402. This allows for monitoring (and measurement) of athletic workouts performed on platform 402. Examples include:
Body weight exercises, for example, stationary running, squats, push-ups (with only hands on platform 402), burpees, skipping, jumps, and the like. In some examples, processing system 430 is configured to identify that individual repetitions of an exercise are performed (for example, an individual push up, jump or skip). In some examples processing system 430 is additionally configured to measure performance metrics for each repetition, for example, based on data attributes extracted from time series force data. This allows, for instance, derivation of metrics representative of jump height and the like.
Object impact exercise, for example, medicine ball workouts. In such exercises, a user interacts with an object, and impacts that object against platform 402 (in the present examples whilst also standing on platform 402). Again, processing system 430 is configured to identify and count repetitions, and preferably performance metrics (for example, impact force).

The load cells may optionally be a GML654 Galoce Robotic Tactile Sensing Load Cell, or similar device, for example, with a 20 KN rating. Those skilled in the art will recognize how appropriate load cells are selected based on attributes such as resolution, dynamic range, and sampling rate. Multiple different load cell types bay be used, with one type selected for optimal sensitivity (to assist in identifying specific motions), and another for optimal sample rate (which may be relevant in accurately identifying peak impacts). In further embodiments, means other than load cells are used to measure force applied to platform 402. This may include technologies that measure strain in platform 402.

In the example of FIG. 4A and FIG. 4B, base member includes a plurality of connector members 405, which are located beneath apertures 406 in platform 402. Each connector member is configured to enable connection of a resistance band, for example, via a carabiner or the like. For example, in some embodiments, each connector member include a loop, latch, cylindrical bar, or the like. The number and location of connector members 405 and apertures 406 varies between embodiments, with the positioning shown in FIG. 4A being an example only. Preferably at least two connector members 405 are provided. These connector members 405 may be provided on base member 403 peripherally outward of platform 402, negating the need for apertures 406. In any event, connector members 405 allows the user to perform resistance exercises via device 400. Tension between the user and one or more resistance bands increases force between platform 402 and base member 403, which is observed in load cells 404. This allows for processing system 430 to identify exercise repetitions, and determine performance metrics of those repetitions (for example, amount of work being performed, time under tension, rate of movement, and so on).

Connector members 405 are preferably provided as opposed pairs, thereby to allows a resistance band for each side of the body. Providing multiple pairs as shown in FIG. 4A allows for a greater range of exercise options, including use of multiple connection points for each resistance band (e.g., to cross over a user's back during resistance push-ups). Furthermore, using multiple connection points on a single resistance band allows for resistance characteristics of a that band to be adjusted, rather than needing to switch between bands or different ratings.

Device 400, as illustrated, also includes a display screen 140 (for example, an LED display). Display screen 140 is coupled to processing system 430, and enables for the display of a range of information. This may include, for example:
Workout instructions. For example, a user is provided with instructions on an exercise that is to be performed, and/or special steps to be undertaken. This is particularly relevant for workout playlists as discussed further below,. whereby a user undertakes a workout involving multiple different exercise types.
A user interface with selection means. In some embodiments, device 400 includes input means, for example, buttons or the like, thereby to facilitate interaction with controls and display screen 140. In one embodiment, platform 402 serves as an input, with different locations (e.g., corners) operating as quasi-buttons, with processing system 430 determining input locations via comparative load cell data values.
Live workout metrics. For example, this may include repetition count, repetition rates, calories burned, calorie burn rate, repetition impact force, average impact force, cumulative impact force, resistance metrics, and so on. In some cases a user can switch between different metric display options, as is common in known workout equipment.

In some embodiments, display screen 140 is a secondary means for input and control of device 400, with a user instead interacting via a smartphone app executing on a mobile device coupled to device 400 via a network connection, as discussed further below.

In the illustrated embodiment, device 400 additionally includes a set of indicator lights 411. These may be substituted for a range of other output devices. These are preferably used to provide any one or more of the following outputs:
A timer, for example, showing a user their temporal progression through a workout.
A force indicator, showing a user the amount of force they are putting into platform 402. This is preferably normalized based on user weight, so that both large and small users are able to experience a dynamic range of output values.
A repetition counter (for example, counting down a number of repetitions for a defined workout).
A metronome, for example, providing a visual indicator for timing of exercises and/or exercise components. This may include, for a resistance exercise, a metronome showing a lift portion, hold portion, release portion, and rest portion.

FIG. 4C illustrates functional components of processing system 430. Processing system 430 is preferably embedded inside base member 403, and includes a microprocessor, which is configured to execute software instructions thereby to perform various key functionalities of device 400. These include:
Processing data received from load cells 404, thereby to determine data values. These may include data values representative of athletic performance.
Delivering a user interface, via an output device. In the illustrated example, a display screen 140 is provided.
Receiving and processing user interface inputs, for example, via buttons, connected devices, through platform 402, and the like.
Executing software instructions representative of one or more predefined workout programs, thereby to, via a display screen: (i) instruct a user to perform athletic activities; and (ii) display data derived from the one or more sensors thereby to provide feedback data regarding athletic performance in an executing one or the one or more predefined workout programs.

In some embodiments, processing system 430 processes a time domain signal, and from that processing extracts data artefacts that are associated with particular signal attributes. For example, in a single platform arrangement, a time domain signal is representative of a cycle of event repetitions (e.g., impact repetitions, body movement repetitions, and/or resistance exercise repetitions). For example:
In the case of a medicine ball workout, each repetition includes: (i) a start component, where a user stands on the platform and the ball rests on the platform; (ii) a lift component, where the user accelerates the ball upwards; (iii) a slam component, where the user accelerates the ball downwards; (iv) a transit component, where the ball is airborne; and (v) an impact component whereby the ball impacts the platform and decelerates to zero velocity.
In the case of a body weight exercise, each repetition includes: (i) a start component where there is a nominal body weight force applied through the platform, (ii) a positive motion component where the force increases and peaks (e.g., during a jump motion); (iii) optionally a zero force component where there is no force applied to the platform (e.g., whilst the user is airborne); and (iii) a negative motion component (e.g., where the user lands from a jump).
In the case of a resistance weight exercise, each repetition includes: (i) a start component where the force through the platform is a local minima, being a force associated with body weight and a minimum level of stretch in resistance bands; (ii) a positive motion component whereby the force progressively increases as tension in the resistance band is increased through an exercise motion; (iii) optionally a peak component, whereby the force stabilizes at an approximate maximum for a period of time (e.g., a user holds in tension at the maximum position of a motion for a prolonged period); and (iv) a negative motion component whereby force progressively decreases as tension in the resistance band lessens, during a motion of return to the start position. It will be appreciated that the timing of each component of the exercise may be relevant for the purposes of instruction and/or assessing performance. In some embodiments, a timer/metronome may be provided to guide movement rate.

In use, a user interacts with the user interface device thereby to activate device 400 (for example, bringing device 400 out of a sleep/dormant mode). The user then selects one of the predefined workout programs, and in some cases is prompted to enter one or more data attributes (for example, age). The workout program then commences, and the user participates in the workout as directed (for example, by slamming the medicine ball repeatedly onto platform 402). Physical values are measured by load cells 404, and processed by processing system 430, which in response causes output via display screen 140 of workout metrics (for example, repetition rate, effort metrics, and so on).

Example workout programs include:
Medicine ball—Cumulative impact force target. A cumulative force target value is defined, and a user seeks to hit that target in the shortest time possible.
Medicine ball—Average impact force target. An average force target value is defined, and a user seeks to maintain an average impact force above that target for the duration of a workout.
Medicine ball—Interval training, whereby a user is instructed in respect of both a “work” period, where there is a focus on maximizing impact force, and a “rest” period, where there is a focus on maintaining a threshold rate of repetitions.
Body weight—Skipping, whereby a user is instructed to perform a skipping workout whilst standing on the platform.
Body weight—Running, whereby a user is instructed to perform a stationary running workout whilst standing on the platform.
Body weight—Jumps, whereby a user is instructed to perform a jumping workout whilst standing on the platform.
Resistance—Bent Over Row, whereby a user stands on the platform, grasps two resistance bands attached to the base (or grasps a bar that connects the resistance bands at their respective proximal ends), and performs a bent over row exercise.
Resistance—Squat, whereby a user stands on the platform, grasps two resistance bands attached to the base (or grasps a bar that connects the resistance bands at their respective proximal ends), and performs a squat exercise.
Resistance—Bench Press, whereby a user lies with their upper back on the platform, grasps two resistance bands attached to the base (or grasps a bar that connects the resistance bands at their respective proximal ends), and performs a bench press style exercise.

It will be appreciated that these are examples only, and that a range of other workout types may be configured. This is particularly the case for resistance exercises, where different length of resistance bands and attachments may be configured to allow a wide range of workouts.

FIG. 4C illustrates functional components of an example processing system 430. Example processing system 430 includes a microprocessor 453, which is configured to execute software instructions 455, which are stored on a memory device. For example, this may include software that operates on an Android operating system.

A user interface module 456 is configured to cause rendering of a graphical user interface via a display screen, which is coupled to processing system 430 via a display/controller interface 452. User interface module controls display of rendered content in response to programming logic and plurality of inputs, including:
Inputs received from a user via interface 452. For example, these may include inputs provided via a touch screen, and/or one or more buttons (and/or, in some embodiments, input delivered by a user's foot input to specific locations on platform 402). Inputs may include, for example: (i) an input that triggers recovery from a “sleep” mode; (ii) an input that selects a workout program from a plurality of displayed workout programs; (iii) inputs that provide parameters relevant to customization of the selected workout program (for example, weight, time, intensity, and the like); (iv) parameter display settings; and (v) workout program controls (for example, pause, stop, restart, and the like).
Input from training program logic module 458, which maintains execution instructions for a plurality of selectable workout programs. For example, upon selection of a workout program by a user, training program logic module 458 provides instructions to the user interface module to configure display of data fields, including fields configured for pre-workout data collection prompts, fields configured to display intra-workout metrics, and fields configured to display post-workout results.
Inputs derived from processing of sensor data received via sensor inputs 451, via a sensor data processing module 457. Sensor data processing module 457 is configured to receive raw inputs from connected sensors, and process those thereby to provide displayable metrics. This may include operations including rounding, averaging, normalizing, combining, and the like. In the present embodiment, storage module 460 stores calculated values in data tables in a storage module 460. These values are used for either or both of: (i) subsequent calculations (for example, where averages are calculated); and (ii) pulling of data for display in display fields rendered on the display screen under control of training program logic module 458.

In some embodiments, a user customization module 459 allows a user to customize various functionalities of processing system 430, for example, by accessing a predefined user account with historical data stored in storage module 460.

In some embodiments, a system link communication module 461 is configured to provide system link functionalities, thereby to enable display of competitive metrics from a plurality of two or more devices. Module 341 is optionally configured to perform either or both of the following:
Transmitting current workout metrics defined by sensor data processing module 457 and/or stored in device 400 to a remote device, which may be a shared display screen or a second similar athletic training device having similar hardware (e.g., a similar processing system 430).
Receiving current workout metrics from an external device (for example, a second similar athletic training device having similar hardware), and providing those metrics for display via user interface module 456 on the display screen.

In relation to the latter, in some embodiments, user interface module 456 and 341 interact thereby to provide user interface options to select a multi-participant workout, in which user interface module 456 configured the user interface to provide data display fields configured to present metrics from one or more other devices, thereby to provide comparison metrics (allowing a user of visually monitor their performance against others).

System link coordination module 461 is coupled to one or more other devices via communications interfaces 454, which may include wired connections and/or wireless connections (e.g., Wi-Fi or Bluetooth).

In further embodiments, the platform is configured to interact with a range of peripheral devices, including (but not limited to): biometric sensors; smart watches; smartphones; smart mirrors (for example, the “MIRROR” home gym), and the like. It will be appreciated that such devices may be configured to interact with the platform thereby to provide instruction, display performance results, and/or gather additional metrics.

In some embodiments, communications interfaces 454 are configured to enable communication with remote computing devices. For example, this may include: computing systems, thereby to enable functions such as configuration and software updates; user mobile devices, thereby to provide additional functionalities based on mobile device app integration; and wearable sensors (for example, via Ant+standard) thereby to enable heartrate and/or other metrics to be communicated to user interface module 456.

In some embodiments, where communications interfaces 454 are configured to enable communication with remote computing devices, device 400 is configured to be operate in conjunction with a smartphone app thereby to facilitate improved access for control and/or configuration. For example, the smartphone is used as a primary user input device for device 400.

In some embodiments, either via a smartphone or directly, device 400 provides output to a Bluetooth speaker or headset, for example, thereby to deliver instructions and/or provide workout feedback.

It should be appreciated that in the above description of exemplary embodiments of the present disclosure, various features of the present disclosure are sometimes grouped together in a single embodiment, FIG., or description thereof for the purpose of streamlining the disclosure and aiding in the understanding of one or more of the various inventive aspects. This method of disclosure, however, is not to be interpreted as reflecting an intention that the claimed present disclosure requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of the present disclosure.

Furthermore, while some embodiments described herein include some, but not other features included in other embodiments, combinations of features of different embodiments are meant to be within the scope of the present disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the present disclosure.

In the description provided herein, numerous specific details are set forth. However, it is understood that embodiments of the present disclosure may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B, which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the present disclosure, those skilled in the art will recognize that other and further modifications may be made thereto without departing from the spirit of the present disclosure, and it is intended to claim all such changes and modifications as falling within the scope of the present disclosure. For example, any formulas given above are merely representative of procedures that may be used. Functionality may be added or deleted from the block diagrams and operations may be interchanged among functional blocks. Steps may be added or deleted to methods described within the scope of the present disclosure.

1. A system configured to enable multiple-activity functional workouts via distributed exercise machines, the system including:
a machine configuration database that contains a plurality of modality data sets, wherein each modality data set includes, for a specific exercise modality, data representative of:
(i) device configuration data executable via a given one of the exercise machines thereby to configure that device to receive physical input representative of the specific exercise modality; and
(ii) processing configuration data executable via a that one of the exercise machines thereby to configure the device to process physical input representative of the specific exercise modality when operated in accordance with the device configuration data;
a playlist selector module, wherein the playlist selector module is configured to enable selection of a defined playlist, wherein each playlist is representative of instructions to deliver a plurality of exercise modalities in a predefined sequence; and
a playlist delivery module, wherein the playlist delivery module is responsive to a selection via the playlist selector module thereby to cause delivery of data via a network to a defined one or more of the distributed exercises devices, thereby to configure those devices to enable execution of the selected playlist.

2. The system of claim 1, wherein configuring the exercise machine to receive physical input representative of the specific exercise modality includes setting parameters for one or more sensor devices.

3. The system of claim 2. wherein the parameters for the one or more sensor devices include sampling rates and/or sensitivity settings.

4. The system of claim 1. wherein configuring the exercise machine to receive physical input representative of the specific exercise modality includes configuring the device to deliver predefined user directions.

5. The system of claim 4, wherein the directions are provided via any one or more of: (i) a display; (ii) lights; (iii) communication via a user smartphone; (iv) audio instructions delivered by a speaker of the device; and (v) audio instructions delivered via a wireless headphones.

6. The system of claim 1, wherein configuring the exercise machine to process physical input representative of the specific exercise modality includes setting parameters for determining any one or more of the following in respect of an exercise of the specific exercise modality:
(i) that an exercise repetition has been performed in accordance with defined requirements;
(ii) a cumulative number of repetitions of the exercise that have been performed in accordance with defined requirements;
(iii) performance attributes associated with a repetition of the exercise performed in accordance with defined requirements;
(iv) cumulative performance attributes of a plurality of the repetitions of the exercise performed in accordance with defined requirements; and
(v) timing attributes for one or more the repetitions of the exercise performed in accordance with defined requirements.

7. The system of claim 6, wherein the device is configured to determine performance attributes associated with a repetition of the exercise performed in accordance with defined requirements, and those performance attributes include any one or more of the following:
(i) a metric representative of physical exertion;
(ii) a metric representative of technical form; and
(iii) a metric representative of adherence to instruction.

8. The system of claim 1. wherein each playlist is representative of instructions to deliver a plurality of exercise modalities: (i) in a predefined sequence; and (ii) in accordance with defined performance metrics.

9. The system of claim 8. wherein the defined performance metrics include tempo of movement.

10. The system of claim 9, wherein the device configuration data causes the device to provide physical output representative of the tempo of movement.

11. The system of claim 1. wherein the devices execute the selected playlist asynchronously in response to an input provided by an individual local user.

12. The system of claim 1, wherein the devices execute the selected playlist synchronously in response to an instruction delivered via the system.

13. (canceled)

14. (canceled)

15. A computer implemented method configured to enable content generation for a system that enables multiple-activity functional workouts via distributed exercise machines, the method including:
maintaining data representative of customizable templates for a plurality of exercise modalities;
providing a user interface that enables a first user to generate a playlist including a plurality of different selected exercise modalities;
providing access via the user interface for the first user to set values for a plurality of variables in respect of respective customizable templates for each of the selected exercise modalities, such that these are associated with the generated playlist;
defining a unique identification code for the generated playlist; and
enabling a second user to: (i) identify the generated playlist; (ii) select the generated playlist; and (iii) provide an instruction to download data associated with the generated playlist to a specific one of the distributed exercise machines designated by the second user, thereby to configure that specific one of the distributed exercise machines to download data associated with the playlist and guide the second user through a multi-modal workout defined by the playlist.

16. The method of claim 15, wherein setting values for a plurality of variables in respect of a given one of the customizable templates for a given one of the selected exercise modalities includes:
setting a number of repetitions of that exercise modality.

17. The method of claim 15, wherein setting values for a plurality of variables in respect of a given one of the customizable templates for a given one of the selected exercise modalities includes: setting a metric related to exercise work that is required to be completed in respect of that exercise modality.

18. The method of claim 15, wherein setting values for a plurality of variables in respect of a given one of the customizable templates for a given one of the selected exercise modalities includes setting one or more of: repetition tempo; intra-repetition tempo; and inter-repetition tempo.

19. The method of claim 18, wherein the user interface enables the first user to define sequencing order for the different selected exercise modalities of the playlist.

20. The method of claim 16, wherein the different selected exercise modalities include two or more of resistance exercises, body weight exercises, and impacting of a portable object onto a surface.
